(19) United States
(12) Patent Application Publication
(10) Pub. No.: US 2022/0104890 A1
(43) Pub. Date: Apr. 7, 2022

(54) OPEN SOURCE ROBOTIC PLATFORM

(71) Applicant: TransEnterix Surgical, Inc., Morrisville, NC (US)

(72) Inventors: Matthew Robert Penny, Holly Springs, NC (US); Stefan Atay, Raleigh, NC (US); Alexander John Maret, Apex, NC (US); Kevin Andrew Hufford, Cary, NC (US); Nicholas J Bender, Raleigh, NC (US); Sevan Abashian, Morrisville, NC (US)

(21) Appl. No.: 17/064,360

(22) Filed: Oct. 6, 2020

(51) Int. Cl. A61B 34/30; B25J 15/00; B25J 19/02

(52) U.S. Cl. CPC A61B 34/30; B25J 9/1689; B25J 19/02; B25J 15/0009

(57) ABSTRACT

An open platform surgical robotic system for use in manipulating and actuating a surgical hand instrument of the type having a handle configured to be grasped by a human hand and actuation members of elements configured to be operated by fingers of a human hand. The system includes a robotic arm including an end effector having a gripper comprising a plurality of digits. The digits are configured to receive the handle of the manual surgical instrument and operate the actuation members.

### TECHNICAL FIELD OF THE INVENTION

The present invention relates to the field of surgical systems using electromechanical drivers to effect movement of medical instruments within a body cavity.

### BACKGROUND

Surgical systems used for robotically-assisted surgery or robotic surgery employ electromechanical drivers to drive movement of surgical devices within a body cavity, typically in response to signals generated when a user moves a user input device. The surgical devices may be surgical instruments having end effectors, and/or they may be steerable lumen devices adapted to receive such surgical instruments (or a combination of such surgical instruments and lumen devices). The surgical devices include actuation elements (e.g. wires, rods or cables) that, when pushed and/or pulled, cause active bending or articulation at the distal end of the surgical device, which is disposed within a patient's body. Motion produced by the electromechanical drivers is used to push and/or pull the actuation elements to produce this bending or articulation.

To use a surgical robotic system, the surgeon must make use of the surgical instruments that have been specially adapted to interface with that surgical system. However, the reality of the surgical field is that surgeons have preferences on the tools that they choose to use to perform a variety of tasks. Sometimes these preferences are a choice between one brand of an instrument and another, and sometimes between one technology and another. For example, to seal a vessel, one surgeon may prefer to use one sealing modality while another would rather use a different sealing modality. Surgeons may also have preferences on instrument handle styles or sizes and any number of different reasons to choose between the tools required to perform desired surgical tasks.

The present invention describes a surgical robotic system capable of using and operating any off-the-shelf hand instruments available to the surgeon.

### BRIEF DESCRIPTION OF THE DRAWINGS

FIG. 1 shows a first embodiment of an open platform surgical robotic arm making use of an anthropometric five-finger gripper hand on a robotic manipulator arm.

FIG. 2A shows a second embodiment of an open platform surgical robotic arm making use of a three-finger gripper hand on a robotic manipulator arm.

FIG. 2B shows a surgical scope, such as a laparoscope or endoscope, mounted held by the gripper hand of FIG. 2A.

FIG. 2C shows a surgical instrument held by the gripper hand of FIG. 2A.

FIG. 3 schematically illustrates bias two fingers of a gripper hand so as to engage and move a handle trigger of a surgical instrument.

FIG. 4A schematically illustrates a gripper hand with a modified digit. FIG. 4B illustrates the used of roller bearings on the modified digit.

FIG. 5A shows a third embodiment of an open platform surgical robotic arm making use of an adaptor plate with actuated members on a robotic manipulator arm.

FIG. 5B shows the adaptor plate of FIG. 5 supporting a surgical instrument.

### DETAILED DESCRIPTION

Disclosed herein is an open-source surgical robotic platform that can be easily adapted to any instrument. It makes use of the common characteristic possessed by every laparoscopic or endoscopic hand instrument provided by any manufacturer, namely that it is already designed to be manipulated by the human hand.

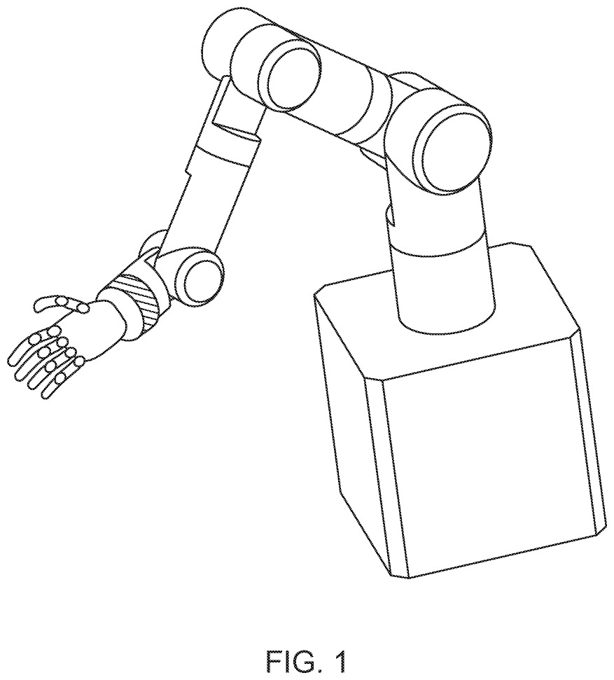

A first primary embodiment of this disclosure is the use of anthropometric robotic “hands” as grippers attached to the end of robotic arms as shown in FIG. 1. These may be used to perform a variety of surgical tasks, including, but not limited to, the following: Instrument Identification, Instrument Assembly, Instrument Exchange, Instrument Holding and Stabilization, Instrument Manipulation in 3D space (autonomous, semi-autonomous, and/or teleoperation), Instrument Actuation via user inputs on the surgeon workstation handles (teleoperation), Surgical Accessory Assembly to an instrument end effector (Clips or Stapling), Instrument Disassembly, Sterilization Tray Packing/Instrument Disinfection measures, Automated Draping (Putting on Gloves), Pre-Op Activities (draping patient bed, etc), Post-Op Activities (Wipe-downs and disposal of trash).

Anthropometric robotic hands have been developed by a variety of different companies. See, for example, the Shunk SUH (https://schunk.com/de_en/gripping-systems/highlights/svh/), Shadow Hand (https://www.shadowrobot.com/products/dexterous-hand/), and the DLR Hand II (http://www.dlr.de/rmc/rm/en/desktopdefault.aspx/tabid-11671/#gallery/286311, each of which is incorporated herein by reference.

When used with a robotic surgical system, the hands or hands can be utilized to grasp and manipulate surgical instrumentation. Modified embodiments will include features providing cleanability suitable for an operating room environment, setting of a maximum grasping strength that can be applied to a surgical instrument for a given task and potentially even the range of motion for each finger so as to allow it to operate a range of surgical instruments. In one such modification, finger mobility is enhanced to allow for the pulling of an actuation trigger or operation of a slide mechanism on an instrument handle. Depending on the type of instrument being grasped, the location of the grasp and the location of the actuators on the instrument, these limitations may need to be addressed to mate with a given tool.

In some implementations, some of the axes of motion or fingers of the robotic hand may be underactuated; that is, the number of actuators may be fewer than the number of degrees of freedom of the finger. Joints may be actively driven, may be passively compliant, or completely free to move. Passive compliance provides the ability to conform to a variety of handle shapes and sizes and reduces system cost and complexity by reducing the number of actuators to accomplish the task. Fingers may move through toggle positions or be bi-stable and “snap” to locked/unlocked positions rather than being controlled through their entire motion. Structural elements of the fingers and hand or pads on the surfaces may deform to comply with the instrument and provide positive support.

New surgical instruments can be conceived of that possess more complex handle interfaces that takes advantage of the anthropometric hands' ability to use other digits more freely than a human hand. For example, it might be desirable to have a feature actuated by the digit of the robotic hand that is analogous to the “little finger” of a human hand, whereas for a typical human user use of that finger would have been more mentally taxing.

Additionally, these anthropometric hands could have features that the human hand is not capable of—for example, the hand may be outfitted with a mechanism for eliminating backlash between the fingers and the surgical tool being grasped. This could be accomplished in any number of ways, but it might be inflation of balloon rings around each digit, the extension of locking or expanding mechanical elements, rotation of a digit in a way to grasp a specific feature on the surgical instrument, the use of more than one digit to expand or fill the open space, or the use of more than one digit to provide locally-opposing forces to each other (such as a pinching grasp). These means and methods could reduce perceived backlash in jaw open/close, for example.

Another advantage of a robotic hand as compared to a human hand is the ability to actuate fingers or pairs of fingers in directions that are non-typical for human hand grasping or more mentally taxing for humans. For example, in instruments having handles that include finger loops (e.g. that the user moves in distal and/or proximal directions to cause a mechanical function of the instrument such as the opening/closing of jaws, the articulating or ending of a part of the instrument), two robotic hand digits could be inserted into a given finger loop and then biased in such a way that one digit is against one edge of the loop and the other digit is against the other edge as illustrated in FIG. 3. This could remove backlash during loop movement (typically jaw open and close) by relying primarily on one digit to close and the other digit to open.

These anthropometric robotic hands could additionally have modified digits to perform tasks specifically related to control and manipulation of surgical instrumentation.

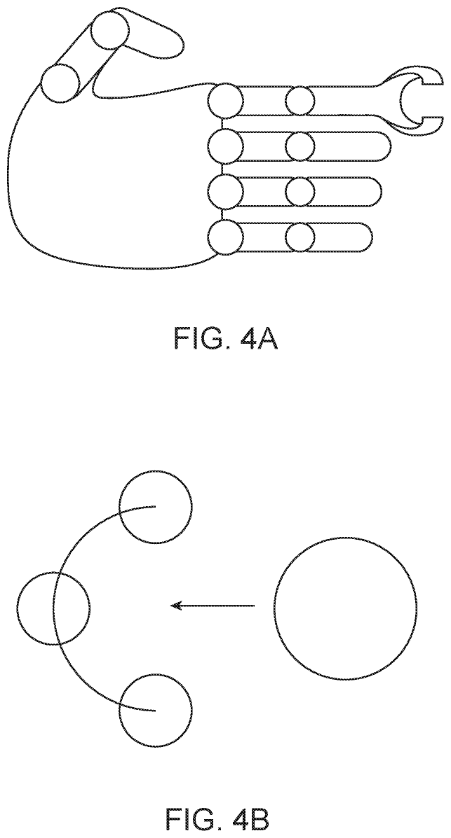

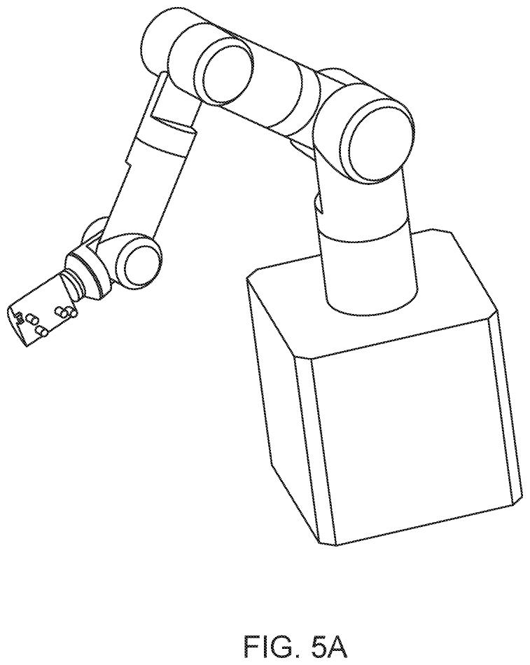

One example of a modified digit is one adapted to allow a finger, e.g. the index finger, to engage with or mate with the surgical instrument shaft. This digit could have a flexible c-shaped guide at the tip, as shown in FIG. 4A, for aligning the surgical instrument to the rest of the anthropometric hand as well as maintaining control of the long shaft of the instrument. Flexible roller bearings might be positioned at points on the guide to serve as the contact points on the guide, as shown in FIG. 4B. The remaining digits could perform the required operations on the instrument handle.

Cameras may be used in conjunction with anthropometric hands to help identify the instrument type, position, orientation and ideal grasping locations. These cameras may be embedded within the anthropometric hands themselves, external to the hands but on the robotic manipulator arms, positioned strategically around the operating room (OR) for different tasks, or any combination of the above. For example, if the camera is embedded in the hand or on the arm, the hand/arm combination can be swept over a location of instruments (OR prep table), using the camera to identify instrument types via barcodes, fiducial markers, shape and/or color recognition or other features. The camera could also determine position and orientation of the instruments, sending that information to the robotic system so that it can be used to determine where and how to grasp a desired instrument. While the goal would be to modify or augment the instruments as little as possible, in another implementation, additional physical adapters could be fitted to the instrument a priori to aid in visual recognition, identification, tracking, and/or pose (position and orientation) determination, or the instruments may be modified to incorporate features to aid in the above.

Alternatively, a camera positioned to view only the OR prep table could also be used to communicate instrument position and orientation to the surgical system by using markers located on the table, or even projected by the camera system itself, for example with a pattern of infrared light or other means of shape determination such as: stereoscopic cameras, laser or white light or infrared light scanning. Once an instrument has been recognized as the desired instrument, the position and orientation coordinates can be communicated to the robotic hand for grasping. A camera on the robot itself or over the OR prep table may also be useful in identifying the location of surgical accessories, for example, clips or staple cartridges. These cartridges could be fixated in such a way as to allow the robotic manipulators to move the instruments into position for accepting a clip or staple cartridge without the need for a surgical assistant.

Other variations may help identify instrument type and location without the need for the camera system at all. For example, if the sterilization trays were designed such that each instrument type could only be installed in one position and orientation for a given instrument type—the robotic system would only need to know the position and orientation of the instrument tray to be able to interpret the location of each instrument within the tray. Another variation may be RFID or wireless near-field-communication that could not only indicate instrument type, but could also be used to determine position and orientation—either through communicating placement of the RFID chip inside the handle, or having sensors such as hall effect magnetic sensors in pre-described positions on the handles.

Additionally, the robotic hands sense of tactile or haptic feedback could also be used to distinguish between two instruments in much the same as a CMM machine is used to take surface measurements of an object being inspected. For example, if the index finger can be extended and moved around the OR prep table in such a way as to come into contact with a given instrument—it could sense that contact. Once contact has been made the finger can be drawn along the instrument, searching for distinguishable features in an attempt to recognize the instrument being touched.

In addition to use the tactile and haptic feedback information for instrument recognition, these features may be more realistically utilized in re-creating forces and sensations at the user interface for the surgeon. For example, grasping force may be sensed at the robotic hand and communicated to the surgeon through a system inside the user interface. Indirect measurements of force such as, but not limited to, motor torques/currents and cable tensions as well as direct measurements from pressure or force sensors at joints, in or on structural or strain members of the finger or on external surfaces of the finger are all considered to be part of this invention. Teaching the robotic hand to hold and manipulate a new instrument could probably be its own disclosure, but for the purposes of this disclosure there are two primary methods. The first is to create an instrument database in which data on the instrument handles can be stored including size, shape and locations of features or functions. These dimensions can be hard-coded to reflect the desired tool to be used. This would be potentially considered a forward kinematic teaching method.

Alternatively, in some cases, robots can be taught to perform repetitive tasks by simply being exposed to a scenario while in a compliant mode. For example, if the robotic hand is in a compliant mode, the instrument can be introduced to the hand and the fingers of the hand externally manipulated by a user to “teach” the robot where to hold the instrument as well as where any relevant buttons, knobs or levers are to perform an array of tasks. The compliant mode could be developed such that it follows a logical template where first the user teaches the robot how to grasp a new tool. Once the grasp has been confirmed, the user can teach the robot how to open and close the jaws for instance and then how to roll the jaws or what button to press for advanced energy if that instrument is capable. These are examples of what can be taught to the robot and this information can then be stored into the instrument database so that it only needs to be taught once. This may be referred to as compliant teaching or reverse kinematic teaching.

Once the robotic hand has been taught how to hold and manipulate an instrument, it will need to know where to place that instrument for operation. This embodiment might use features of the type described in U.S. Pat. No. 9,707,684, which is incorporated by reference, to sense and remember the fulcrum location of the surgical trocar through which the instrument is positioned in the body, or could also use a calculated or measured remote center of motion based on trocar insertion marks, such as those used on other surgical robotic platforms. Cameras on the arm or above the patient could be used to determine the orifice of the trocar, in case of trocar movement after the instrument is removed. This information can be communicated to the robot to facilitate instrument insertion into the abdomen.

Finally, this embodiment will map the movement of the robotic anthropometric hands to a surgeon interface that is capable of collecting surgeon inputs and mapping those inputs to desired motions at the robotic hand for input into the off-the-shelf tools.

This embodiment might use drapes in the form of off-the-shelf latex gloves to cover the robotic arm hands and arms to create separation between sterile and non-sterile components. The drapes may be extended somewhat to provide additional coverage, much like ultrasound probes are draped in the OR. Alternatively, a surgical drape might be included with a distal end having a plurality of discrete sleeves extending from a common “palm” region, with each sleeve positionable over a separate one of the digits or grasping linkages.

The hands could potentially be taught to “self-drape” in much the same way as a human would install sterile gloves on his or her own hands.

Additionally, these robotic hands could be used to prepare instruments or the OR for surgery. For instruments that need assembly, the robotic hands could be used in combination to align and assemble instrumentation. They could also be used to disassemble and re-pack the sterilization trays at the conclusion of a case.

Alternative embodiments make use of alternative robotic grippers or hands that may or may not be anthropometric. Such grippers have been used on high-speed assembly lines or in manufacturing environments for decades. These gripper and arm combinations are used, in combination with cameras to identify the shape and orientation of an object to be grasped, pick up the object and re-place it in a desired orientation and position for the next down-stream process. Some examples of non-anthropometric grippers are the Schunk SDH multi-jointed 3-finger gripping hand (https://schunk.com/hu_en/gripping-systems/series/sdh/) and the Barrett Hand (http://www.barrett.com/products-hand.htm), each of which his incorporated by reference.

These grippers feature 3 functional grasping linkages and up to 7 degrees of freedom to adapting to large and small objects, cylinders, etc. The “finger-tips” also feature tactile sensors to determine pressure which can be used as verification of a “good” grasp or feedback on the consistency of the object being grasped. For example the system might confirm a “good” grasp if all pressure sensors that should contact an instrument when properly gripped detect pressure above a predetermined threshold. Feedback from the sensors might then be monitored through the procedure, so that reductions in sensed pressure (and particularly reductions to below a predetermined threshold) can trigger an alert to a user to inspect the interface between the grippers and instrument to ensure it is secure.

Features and uses described above for the anthrometric may be likewise used for the three-fingered hand.

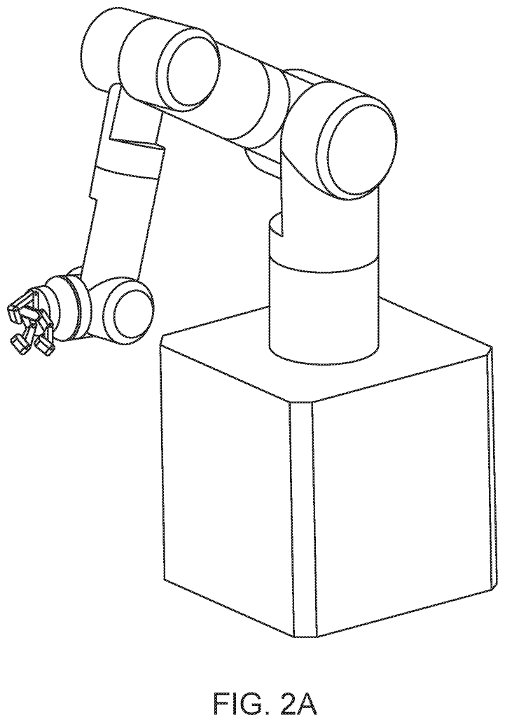

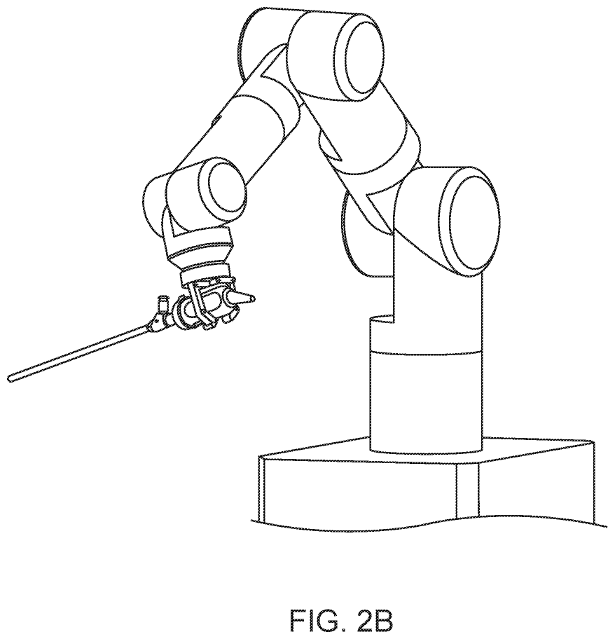

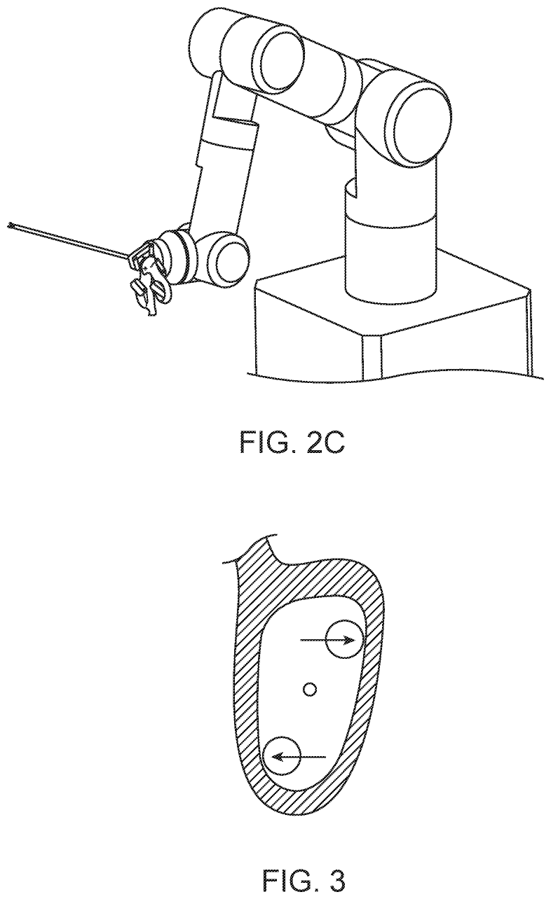

In one exemplary embodiment, a gripper/graspers having a “three fingered hand” type of configuration can be positioned on a robotic arm as shown in FIG. 2A and used in a surgical robotic system. The design of such grippers makes them particularly suitable to picking up a variety of laparoscopic instruments (FIG. 2B), cameras (FIG. 2C) and tools.

The disclosed grippers possess certain advantages over those found on the human hand. For example, ranges of motion including the wrist roll movement relative to an axis of the supporting robotic arm member may not be as limited as a human hand. Additionally, these hands will not fatigue as operators' hands may during long procedures. This might reduce the need for certain instrument features such as ratcheting handles and or roll knobs, and thus enable the surgical robotic company to offer de-featured instrumentation in some cases for a reduced price or elongated instrument life when compared to off-the-shelf instruments.

The end effector of the robotic manipulator arm might include additional actuators oriented to actuated other actions or degrees of freedom on the instrument, such as roll knobs, latches, energy actuation features such as buttons, sliders etc, mode switches etc, or camera features such as zoom, focus, color mode, etc.

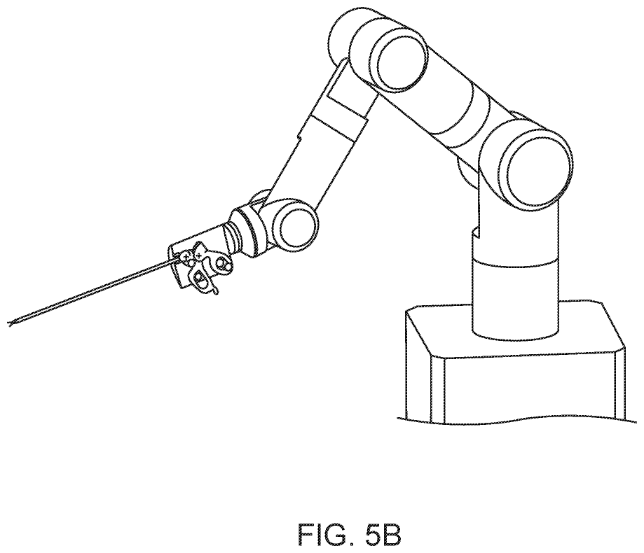

Alternative embodiments differ from the embodiments described above in that they do not use anthropometric or non-anthropometric hands to grasp and hold various instruments, but instead equip the robotic arm with actuators (which may be rotary and/or linear). Referring to FIG. 5A, sterile adapters may be positioned between the actuators and the instrument and include a plurality of mechanical elements (shown as pins, of which any or all may be moveable by the actuators) that transfer the motion between the actuators and the instruments, in addition to a feature which constrains the shaft of the instrument. This constraining/retaining feature may not be necessary, although it relieves some loads from the shaft. The embodiment may be configured by moving the pins into an alternate configuration in which the loads on the instrument and handles are countered by other pins. The number, orientation and direction of motion of the pins will vary. These mechanical elements would be positioned to receive and actuate off-the-shelf instruments as shown in FIG. 5B, preferably without modification, and use each such instrument through its own user interface (handle). While in FIG. 5A the moveable pins are carried by the end effector of the manipulator arm, other embodiments might include a separate adapter plate with moveable pins that can be then attached to the end effector of the robot. The system may be provided with various adaptors, each configured to transfer the motion from the actuators to a particular type of instrument handle. This way, the system enables true open-source capability to allow the surgeon and hospital to continue to use the instruments and value chains that they prefer.

In a variation of this embodiment, the adaptor may be configured to access the hand instruments from both sides, from alternate sides, top and/or bottom in order to appropriately secure the instrument on the arm and engage the actuation features (handles, roll knob, etc) of the instrument.

These alternative embodiments will include the primary feature sets of holding and manipulating off-the-shelf surgical instruments disclosed with respect to the initial embodiments, but might be configured without features allowing them to pick-up instruments by themselves. In such examples, a member of the operating room staff such as a scrub tech or nurse will position the instrument handle within the robotic adapter in order for the tool to be installed on the robotic manipulator arm.

In each embodiment of the open-source gripper attached to the robotic arm, the surgeon control of the gripper and arm will be executed through a remote user interface. The user interface will have a proprietary user input device (similar to a laparoscopic handle) whose ranges of motion and variety of input methods (buttons and levers and knobs) will be mapped to control the open-source gripper. As such, the open-source gripper will not be controlled through typical human hand manipulation, as is achieved with the data glove device and others. Instead, the gripper's movements will be customized for each intended laparoscopic device and mapped to a separate common user interface that may or may not have the same degrees of freedom or input methods as the handle itself. Therefore, it is conceivable that the surgeon's hands may operate the common remote user interface in a different way than the surgeon hand would typically operate the laparoscopic device attached to the robotic gripper. This disconnect between surgeon hand movement and robotic hand movement is believed to be unique and different than the ways that robotic hands are currently implemented in adjacent fields.

We claim:

1. An open platform surgical robotic system for use in manipulating and actuating a surgical hand instrument of the type having a handle configured to be grasped by a human hand and actuation members of elements configured to be operated by fingers of a human hand, the system comprising:
a robotic arm including an end effector having a gripper comprising a plurality of digits, the digits configured to receive the handle of the manual surgical instrument and operate the actuation members.

2. The system of claim 1 where the gripper includes 5 digits.

3. The system of claim 2 wherein the gripper is anthropometric.

4. The system of claim 1 wherein the gripper includes 3 digits.

5. The system of claim 1, wherein the gripper is configured to receive instruments handles of various shapes and configurations.

6. The system of claim 1, further including a surgical drape positionable covering the robotic arm, the surgical drape including a plurality of sleeves, each positionable over a corresponding one of the digits.

7. The system of claim 1, further including a plurality of pressure sensors positioned on the digits to detect contact forces between the digits and the handle.

8. The system of claim 7, wherein pressure sensors are positioned on the digits to detect contact forces between the digits and the actuation members of the handle.

9. An open platform surgical robotic system for use in manipulating and actuating a surgical hand instrument of the type having a handle configured to be grasped by a human hand and actuation members of elements configured to be operated by fingers of a human hand, the system comprising:
a robotic arm including an end effector having an adaptor plate having a plurality of drive members and a plurality of actuators operatively associated with the drive members, the drive members driveable by the actuators to operate the actuation members of the surgical instrument.

10. The system of claim 9, further including stabilization features on the end effector for stabilizing the instrument relative to the adaptor plate.

11. The system of claim 9 wherein the adaptor plate is configured to receive instruments handles of various shapes and configurations.

12. The system of claim 9 further including a further including a plurality of pressure sensors positioned on the drive members to detect contact forces between the drive members and actuation members.
